(19) United States
(12) Patent Application Publication
(10) Pub. No.: US 2023/0190152 A1
(43) Pub. Date: Jun. 22, 2023

(54) APPARATUS AND METHOD FOR NON-INVASIVELY MEASURING VENOUS BLOOD ANALYTES

(71) Applicant: EDWARDS LIFESCIENCES CORPORATION, Irvine, CA (US)

(72) Inventor: Julio Agustin Isla Garcia, Irvine, CA (US)

(21) Appl. No.: 18/162,164

(22) Filed: Jan. 31, 2023

Related U.S. Application Data
(63) Continuation of application No. PCT/US2021/041665, filed on Jul. 14, 2021.
(60) Provisional application No. 63/059,343, filed on Jul. 31, 2020.

(51) Int. Cl. A61B 5/145; A61B 5/00

(52) U.S. Cl. CPC A61B 5/14546; A61B 5/0095; A61B 5/6803; A61B 5/7221; A61B 2562/146

(57) ABSTRACT

A system for measuring an analyte within a cerebral venous blood flow is provided. The system includes an excitation light source, a light delivery element, an ultrasound receiving mechanism, and a controller. The excitation light source is configured to produce one or more wavelengths operable to cause an analyte to photoacoustically produce at least one ultrasonic signal. The light delivery element is disposed on a posterior region of a human head, and receives light from the light source. The ultrasound receiving mechanism is disposed on an anterior region of the head. The mechanism includes at least one ultrasonic receiver. The controller is configured to control the excitation light source to selectively produce the excitation light, and to measure an amount of the analyte present within the cerebral venous blood flow.

This application claims the benefit of PCT/US2021/041665, filed Jul. 14, 2021, which claims priority to U.S. Patent Application Ser. No. 63/059,343, filed Jul. 31, 2020, the entireties of each of which are hereby incorporated by reference.

### BACKGROUND OF THE INVENTION

### 1. Technical Field

The present disclosure relates to systems and methods for non-invasively measuring analytes within mammalian blood vessels in general, and to systems and methods for non-invasively measuring analytes within mammalian blood vessels that use photometric and acoustics means in particular.

### 2. Background Information

Healthcare providers often monitor one or more physiological characteristics of a patient during diagnosis and treatment. A wide variety of monitoring devices have been developed to meet this need and have become an indispensable part of modern medicine. For example, it is known that determining the concentration of certain blood analytes (e.g., oxyhemoglobin, deoxyhemoglobin, etc.) within a subject's blood can facilitate the determination of clinical parameters (e.g., oxygen saturation, etc.), which in turn can provide insight into the subject's respiratory and/or cardiac function. Deviation from normal or expected values may alert a clinician to the presence of a particular physiologic condition. Some known monitoring techniques require an invasive arterial catheter cannulated into a subject's arterial bloodstream. Such invasiveness techniques may cause the patient discomfort, injury, and/or inconvenience. Hence, it would be desirable to provide a non-invasive system and/or method operable to measure analytes within the blood vessels of a subject.

### SUMMARY

According to an aspect of the present disclosure, a system for measuring one or more analytes within cerebral venous blood flow is provided. The system includes an excitation light source, a light delivery element, an ultrasound receiving mechanism, and a controller. The excitation light source is configured to selectively produce one or more wavelengths of excitation light that are operable to cause an analyte present within venous blood flow to photoacoustically produce at least one ultrasonic signal. The light delivery element is configured to be disposed on a posterior region of a human head, and to receive the excitation light from the excitation light source. The light delivery element has a scalp side surface and a plurality of protruding elements extending out from the scalp side surface. The protruding elements are configured as light conduits, and are spaced apart from one another. The ultrasound receiving mechanism is configured to be disposed on an anterior region of the human head. The mechanism includes at least one ultrasonic receiver disposed to sense the at least one photoacoustically produced ultrasonic signal and to produce a sensed signal representative of the photoacoustically produced ultrasonic signal. The controller is in communication with the excitation light source and the ultrasound receiving mechanism, and a memory storing instructions. The instructions when executed cause the controller to: control the excitation light source to selectively produce the one or more wavelengths of excitation light; and measure an amount of the analyte present within the cerebral venous blood flow using the sensed signals produced by the at least one ultrasonic receiver, the sensed signals representative of the photoacoustically produced ultrasonic signals.

In any of the aspects or embodiments described above and herein, the light delivery element may be configured to distribute the excitation light in a substantially uniform manner through the protruding elements.

In any of the aspects or embodiments described above and herein, the light delivery element may include a plurality of side surfaces, and a back surface opposite the scalp side surface, and a fully reflective material attached to the plurality of side surfaces and to the back surface, the fully reflective material disposed to reflect the excitation light internally within the light delivery element.

In any of the aspects or embodiments described above and herein, the light delivery element may include a semi-reflective material attached to the scalp side surface, the semi-reflective material disposed to reflect less than all of the excitation light incident to the semi-reflective material internally within the light delivery element.

In any of the aspects or embodiments described above and herein, the light delivery element may include a plurality of side surfaces, and a back surface opposite the scalp side surface, the back surface have a curvilinear shape configured to direct excitation light incident to the back surface internally within the light delivery element towards the protruding elements.

In any of the aspects or embodiments described above and herein, the light delivery element may include a plurality of side surfaces, and a back surface disposed at an acute angle relative to the scalp side surface, and a diffusive reflector disposed proximate the back surface, the diffusive reflector configured to reflect excitation incident to the diffusive reflector internally within the light delivery element towards the protruding elements in a uniform distribution.

In any of the aspects or embodiments described above and herein, the light delivery element may have an incidence region that includes the plurality of protruding elements, wherein the incidence region is configured to permit alignment with at least a portion of a superior sagittal sinus, and/or at least a portion of a transverse sinus.

In any of the aspects or embodiments described above and herein, the incidence region may be configured to permit alignment with at least a portion of the superior sagittal sinus, and/or at least a portion of a left transverse sinus or at least a portion of a right transverse sinus.

In any of the aspects or embodiments described above and herein, the incidence region may be configured to permit alignment with at least a portion of the superior sagittal sinus, and/or at least a portion of both the left transverse sinus and the right transverse sinus.

In any of the aspects or embodiments described above and herein, the system may include a head apparatus configured to support the light delivery element and ultrasound receiving mechanism, the head apparatus configured to position the light delivery element on a posterior region of a human head, and configured to position at least a part of the ultrasound receiving mechanism on an anterior region of the human head, the light delivery element having an incidence region configured to permit alignment with at least a portion of a superior sagittal sinus, and at least a portion of a left transverse sinus or at least a portion of a right transverse sinus.

In any of the aspects or embodiments described above and herein, the light delivery element may have a plurality of incidence subregions, the incidence subregions disposed to permit alignment with at least a portion of a superior sagittal sinus, and/or at least a portion of a transverse sinus. The instructions when executed may cause the controller to direct the excitation light to select ones of the plurality of incidence subregions.

According to another aspect of the present disclosure, a method for measuring one or more analytes within cerebral venous blood flow of a subject is provided. The method includes the steps of: a) using an excitation light source to produce one or more wavelengths of excitation light, the one or more wavelengths of excitation light operable to cause an analyte present within venous blood flow to photoacoustically produce at least one ultrasonic signal; b) directing the excitation light to a posterior region of a human head using a light delivery element, the excitation light oriented to be incident to at least a portion of the superior sagittal sinus of the subject, at least a portion of a left transverse sinus, or at least a portion of a right transverse sinus of the subject; c) using an ultrasound receiving mechanism to sense ultrasonic signals from an anterior tissue region of the subject, and to produce sensed signals representative of the sensed ultrasonic signals; and d) using a controller in communication with the excitation light source and the ultrasound receiving mechanism to measure an amount of the analyte present within the at least a portion of the superior sagittal sinus of the subject, the at least said portion of said left transverse sinus, or the at least said portion of said right transverse sinus of the subject using the sensed signals produced by the ultrasound receiving mechanism.

In any of the aspects or embodiments described above and herein, the step of directing the excitation light to the posterior region of the human head may include directing at least a portion of the excitation light to be incident to the at least said portion of the superior sagittal sinus of the subject, the at least said portion of said left transverse sinus, or at least said portion of said right transverse sinus in a manner that creates an acoustic aperture directed substantially toward the ultrasonic receiving mechanism.

In any of the aspects or embodiments described above and herein, the step of directing the excitation light to the posterior region of the human head using the light delivery element, may include directing the excitation light to the posterior region in a substantially uniform distribution of light intensity.

In any of the aspects or embodiments described above and herein, the light delivery element may have a plurality of incidence subregions, and the step of directing the excitation light to the posterior region of the human head using the light delivery element, may include directing the excitation light to at least a first one of the plurality of incidence subregions, and not directing the excitation light to at least a second one of the plurality of incidence subregions.

In any of the aspects or embodiments described above and herein, the method may further include directing at least a portion of the excitation light to be incident to the at least a portion of the superior sagittal sinus of the subject, at least a portion of the left transverse sinus, or at least a portion of the right transverse sinus in a manner that creates an acoustic aperture directed substantially toward the ultrasonic receiving mechanism.

In any of the aspects or embodiments described above and herein, the step of using the controller to measure the amount of the analyte may include determining a signal to noise ratio (SNR) for each of a plurality of channels.

In any of the aspects or embodiments described above and herein, the step of using the controller to measure the amount of the analyte may include measuring the amount of the analyte with the sensed signals from less than all of the channels.

In any of the aspects or embodiments described above and herein, the step of using the controller to measure the amount of the analyte may include determining a time delay of the sensed signals between the respective channels.

In any of the aspects or embodiments described above and herein, the step of using the controller to measure the amount of the analyte may include using the sensed signals to identify at least one of the superior sagittal sinus, the left transverse sinus, or the right transverse sinus.

The foregoing features and elements may be combined in various combinations without exclusivity, unless expressly indicated otherwise. These features and elements as well as the operation thereof will become more apparent in light of the following description and the accompanying drawings. It should be understood, however, the following description and drawings are intended to be exemplary in nature and non-limiting.

### BRIEF DESCRIPTION OF THE DRAWINGS

FIG. 1 is a diagrammatic view of an embodiment of the present disclosure system, with a side view of a head apparatus mounted on a subject's head.

FIG. 2 is a diagrammatic view of the embodiment of the present disclosure system shown in FIG. 1, with a top view of a head apparatus mounted on a subject's head.

FIG. 3 is a diagrammatic view showing the relevant position of the superior sagittal sinus and the transverse sinuses.

FIG. 4 is a diagrammatic view of a light delivery element incidence region embodiment.

FIG. 5 is a diagrammatic view of a light delivery element incidence region embodiment.

FIG. 6 is a diagrammatic view of a light delivery element incidence region embodiment.

FIG. 7 is a diagrammatic view of a light delivery element incidence region embodiment.

FIG. 8 is a diagrammatic view of a light delivery element incidence region embodiment.

FIG. 9 is a diagrammatic view of a light delivery element incidence region embodiment.

FIG. 10 is a diagrammatic side view of a light delivery element embodiment.

FIG. 10A is a diagrammatic top view of the light delivery element embodiment shown in FIG. 10.

FIG. 11 is a diagrammatic side view of a light delivery element embodiment.

FIG. 11A is a diagrammatic top view of the light delivery element embodiment shown in FIG. 11.

FIG. 12 is a diagrammatic side view of a light delivery element embodiment.

FIG. 12A is a diagrammatic top view of the light delivery element embodiment shown in FIG. 12.

FIG. 13 is a diagrammatic side view of a light delivery element embodiment.

FIG. 13A is a diagrammatic top view of the light delivery element embodiment shown in FIG. 13.

FIG. 14 is a diagrammatic side view of a light delivery element embodiment.

FIG. 14A is a diagrammatic top view of the light delivery element embodiment shown in FIG. 14.

FIG. 15 is a diagrammatic side view of a light delivery element embodiment.

FIG. 16 is a diagrammatic view of a light delivery element embodiment in communicating with a steering mechanism.

FIG. 17 is a diagrammatic view of a light delivery element embodiment in communicating with a steering mechanism.

FIG. 18 illustrates a graph of photoacoustic signal (Y-axis) versus time (X-axis).

FIG. 19 is a diagrammatic illustration of a present disclosure system embodiment.

FIG. 20 is a flow chart describing a present disclosure system embodiment.

### DETAILED DESCRIPTION

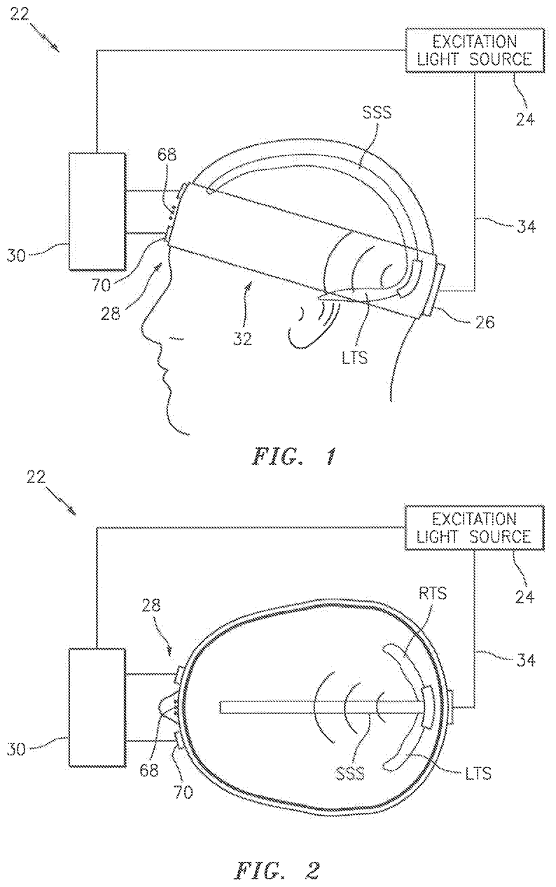

Referring to FIGS. 1 and 2, the present disclosure is directed to a system 22 and method for non-invasively measuring the concentration of one or more analytes present within venous blood flowing within cerebral venous blood vessels. Analytes that may be measured with the present disclosure include, but are not limited to, oxyhemoglobin (“HbO2”), deoxyhemoglobin (“Hb”), methemoglobin, carboxyhemoglobin, carbon monoxide, and the like. To facilitate the description herein, the analytes may be referred to hereinafter as HbO2 and Hb, but as stated the present disclosure is not limited thereto. Analyte measurements may be used to determine the concentration of respective analytes, as well as additional physiological/hemodynamic parameters, such as oxygen saturation.

A system 22 according to present disclosure may include an excitation light source 24, a light delivery element 26, an ultrasound receiving mechanism 28, and a controller 30 in communication with the excitation light source 24 and the ultrasound receiving mechanism 28. In some embodiments, the controller 30 may be in communication with the light delivery element 26. In some embodiments, the present disclosure may include an apparatus that can be placed on a subject's head (referred to hereinafter as a “head apparatus 32”), configured to position the excitation light source 24 and at least a portion of the ultrasound receiving mechanism 28 relative to the subject's head.

The excitation light source 24 is configured to produce light at a plurality of predetermined wavelengths. In some embodiments, the excitation light source 24 may include a plurality of lasers. Each laser may be configured to emit a light beam at a wavelength of light different from that emitted from the other lasers. The wavelengths are chosen based on their ability to produce a photoacoustic effect (sometimes referred to as an “optoacoustic effect”) when light emitted by the laser is sufficiently absorbed by a target analyte. The term “photoacoustic effect” as used herein refers to the phenomenon that occurs when light at a particular wavelength is presented to and absorbed by the target analyte, thereby causing an increase in kinetic energy of the target analyte and consequent pressure response from the analyte in the form of an acoustic wave. The aforesaid acoustic waves may be referred to hereinafter as ultrasonic signals. In some embodiments, the excitation light source 24 may be located remotely from the subject and the light produced by the excitation light source 24 may be communicated to the subject's scalp via one or more optical fibers 34 or other light conduits. The optical fibers 34 may be configured to communicate the excitation light directly to the subject's skin, or the optical fibers 34 may be in communication with one or more light delivery elements 26, such that the excitation light must pass through the light delivery element(s) 26 prior to being incident to the subject's skin. The system 22 may be configured such that the excitation light source 24 is disposed remotely from the head apparatus 32 or the head apparatus 32 may be configured to include the excitation light source 24.

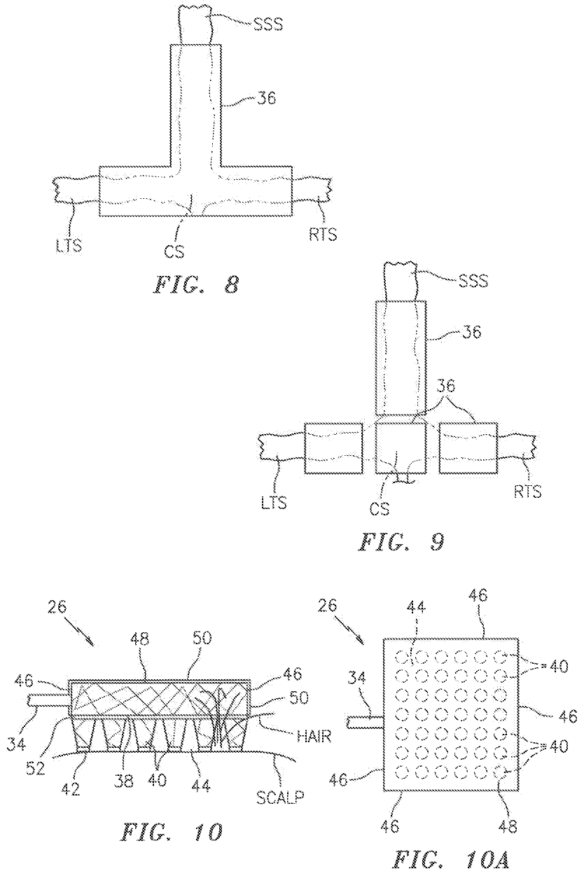

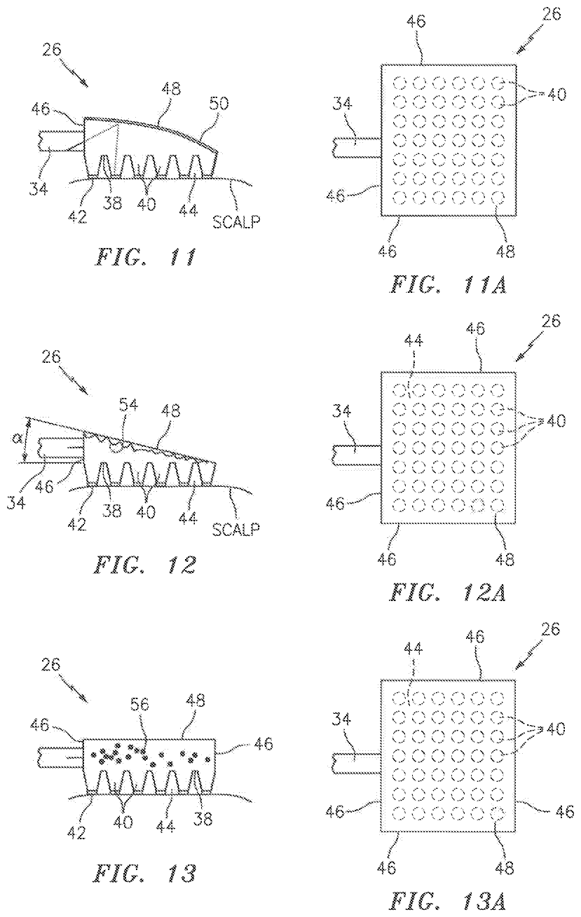

Referring to FIGS. 4-14A, the light delivery element 26 is configured to be disposed contiguous with a subject's scalp and to deliver excitation light to the subject's scalp. The present system 22 contemplates that light delivery element 26 may be disposed relative to the subject's scalp in a plurality of different ways. Non-limiting examples include a light delivery element 26 that is independent of the head apparatus 32, or one that is attached to the head apparatus 32, or one that is incorporated with the head apparatus 32. The light delivery element 26 is configured to deliver excitation light to the subject's head at the posterior of a subject's head. The term “posterior of a subject's head” as used herein refers to that portion of the subject's head having a line of sight with the anterior of the subject's head; e.g., the subject's forehead.

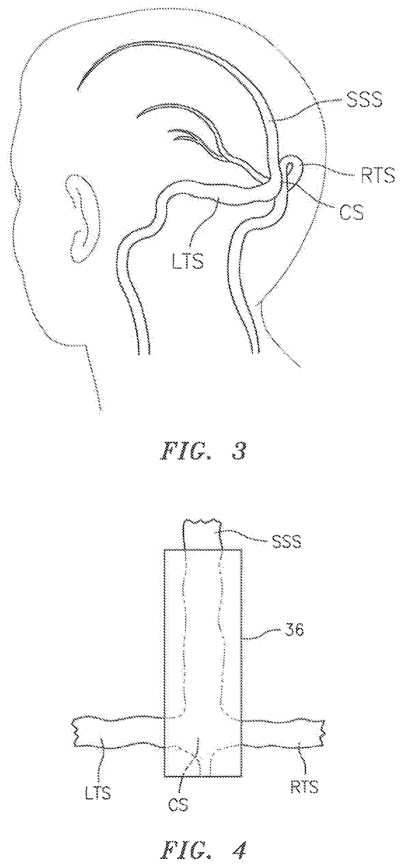

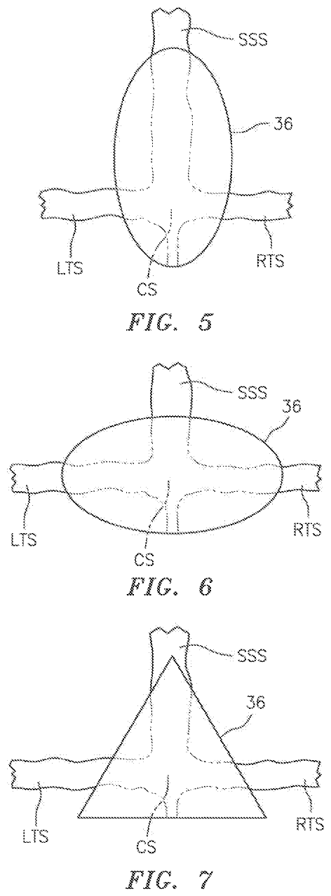

In preferred embodiments, the light delivery element 26 is configured to deliver excitation light to a portion of the subject's scalp aligned with at least a portion of the superior sagittal sinus (“SSS”), at least a portion of the left transverse sinus (“LTS”), and/or at least a portion of the right transverse sinus (“RTS”) (e.g., see FIG. 3). The term “align” or “alignment” as used here refers to the spatial orientation between the light delivery element 26 and the SSS, LTS, and/or RTS. A light delivery element 26 “aligned” with the SSS, LTS, and/or RTS will produce excitation light incident to the SSS, LTS, and/or RTS. Preferably, the alignment is such that the excitation light incident to the SSS, LTS, and/or RTS produces ultrasonic signals having wavefronts that are substantially parallel to at least some of the ultrasonic receivers 68 of the ultrasound receiving mechanism 28 (described hereinafter), and the greatest intensity of the ultrasonic signals is directed substantially toward the ultrasonic receivers 68. In this configuration, the SSS, LTS, and/or RTS (or portions thereof) act as an acoustic aperture(s) that directs ultrasonic signals toward the ultrasonic receiver(s) 68). The LTS and the RTS may be collectively referred to herein as the transverse sinus or “TS”. The SSS extends along a line that generally extends between the posterior and anterior regions of a subject's head. The SSS, LTS and RTS meet at the confluence of the sinuses (“CS”), with the LTS extending left, laterally away from SSS, and the RTS extending right, laterally away from SSS (as viewed from the posterior of the skull). In preferred embodiments, the light delivery element 26 has an incidence region 36 configured to align with a portion of the SSS, or the LTS, or the RTS, or combinations thereof (which may include the CS) disposed at the posterior of a subject's head. The term “incidence region” as used herein refers to the geometry of the light delivery element 26 that delivers excitation to the posterior area of the subject's head during operation of the system 22. FIG. 4 diagrammatically illustrates a light delivery element 26 incidence region 36 configured to align with the CS and a length of the SSS. The diagrammatic illustration of FIG. 4 depicts the light delivery incidence region 36 as generally rectangular in shape. The present disclosure light delivery elements 26 are not limited to this rectangular shape and may include any shape having a length substantially longer than its width. For example, FIG. 5 illustrates a light delivery element 26 incidence region 36 configured to have an elliptical shape with a major axis that aligns with the confluence of the sinuses and a length of the SSS. FIG. 6 diagrammatically illustrates a light delivery element 26 incidence region 36 configured (e.g., elliptical shape) to align with the confluence of the sinuses and a length of the LTS and a length of the RTS (i.e., the major axis aligns with the confluence of the sinuses and a length of the LTS and the RTS). FIGS. 7-9 diagrammatically depict light delivery incidence regions 36 configured to align with the confluence of the sinuses, a portion of the SSS, a portion of the LTS, and a portion of the RTS; e.g., T-shaped, or triangularly shaped, or the like.

A person of skill in the art will recognize that light entering tissue from the scalp (at intensities acceptable under applicable standards) will randomly scatter at tissue depths greater than about one millimeter. Preferred embodiments of the present disclosure that include a light delivery element 26 configured to align with portions of the SSS, LTS, or RTS, and combinations thereof, account for the scattering by directing the excitation light from the light delivery element 26 to regions (i.e., SSS, LTS, TTS) where a significant venous blood source is disposed; hence, they deliver the excitation light to regions having a greater amount of venous blood and therefore the analyte(s) of interest. The location of the SSS, LTS, and the RTS in the posterior region of the subject's head relative to the anterior forehead of the subject, also enable the SSS, LTS, and RTS to function as an acoustic aperture that facilitates the production of photoacoustic signals using present disclosure light delivery element 26 configurations as described herein. To be clear, however, a light delivery element 26 configured to align with portions of the SSS, LTS, or RTS, and combinations thereof is a preferred embodiment of the present disclosure, but is not required.

Some embodiments of the present disclosure light delivery element 26 are configured to spatially control the intensity distribution of the light emitted from the light delivery element 26; e.g., configured to provide a spatially uniform light intensity distribution, or configured to provide a defined non-uniform light intensity distribution, or configured to provide a light intensity distribution that can varied by steering the light input to the light delivery device, or combinations thereof. Controlling the light intensity distribution to the scalp surface provides several advantages. A person of skill will recognize that safety regulations typically limit the total amount of energy to which tissue can be exposed (e.g., via incident light), and/or the intensity level of incident light to which tissue can be exposed in a given period of time. Light delivery element 26 embodiments of the present disclosure make it possible to deliver a maximum permissible total amount of incident light energy and/or light intensity in a given period of time in a variety of different ways.

Some present disclosure light delivery elements 26 may be configured to provide a substantially uniform distribution of light intensity throughout the incidence region 36 of the light delivery element 26. The uniform distribution may allow for a greater collective light energy transfer through the scalp without exceeding relevant safety standards. In turn, the greater collective light energy transfer can yield a substantial increase in signal to noise ratio (“SNR”) for the same or lower amount of optical power.

FIGS. 10-13 illustrate non-limiting examples of light delivery elements 26 may be configured to provide a substantially uniform distribution of light intensity throughout the incidence region 36 of the light delivery element 26. The light delivery element 26 shown in FIGS. 10 and 10A is configured to have a plurality of elements protruding outwardly from a scalp side surface 38 (“protruding elements 40”). The protruding elements 40 each have a contact surface 42 located at a distal end and are light conduits that are configured to transfer light from the light delivery element 26 to the subject's scalp at the contact surface. The protruding elements 40 are spaced apart from one another, thereby creating open spaces or “voids” disposed between adjacent protruding elements 40. The aforesaid open spaces/voids may be referred to herein as trenches 44. The protruding elements 40 may be orthogonally organized, or disposed in a predetermined non-orthogonal pattern, or they may be randomly disposed on the scalp side surface 38. The present disclosure is not limited to any particular spatial configuration of protruding elements 40. When applied to a subject's scalp covered with hair, the protruding elements 40 advantageously provide light conduits to the subject's scalp and substantially avoid photometric interference that may otherwise occur by a substantial amount of hair being disposed between the light delivery element 26 and the subject's scalp.

The light delivery element 26 embodiment shown in FIGS. 10 and 10A is a body of light conductive material (e.g., glass materials, polycarbonate materials, cyclic olefin polymers, NAS, fused silica, N-BK7 (optical borosilicate-crown glass produced by Schott AG of Mainz, Germany), and the like) having a plurality of side surfaces 46 (e.g., four), a back surface 48, and a scalp side surface 38. A light conduit (e.g., one or more optical fibers 34) operable to communicate light from the excitation light source 24 to the light delivery element 26 is shown connected to one of the side surfaces 46 of the light delivery element 26. In this embodiment, a fully reflective material 50 is disposed on each side surface 46 and the back surface 48. The fully reflective material 50 can be disposed on the aforesaid surfaces 46, 48 in any manner (e.g., adhered to, or coated on, etc.), so that the fully reflective material 50 is facing the interior of the light delivery element 26. The term “fully reflective” is used herein to mean that substantially all of any light incident to the fully reflective material (i.e., light traveling internally within the light delivery element 26) is reflected from the fully reflective material 50, and therefore reflects back within the light delivery element 26 body and does not pass through the fully reflective material 50 (and therefore does not exit the light delivery element 26). A semi-reflective material 52 is attached to (or coated onto) a portion or all of the scalp side surface 38. The term “semi-reflective” is used herein to mean that less than all of any light incident to the semi-reflective material (i.e., light traveling internally within the light delivery element 26) is reflected from the semi-reflective material 52; i.e., a portion of the incident light reflects back within the light delivery element 26 body, and a portion of the incident light passes through the semi-reflective material 52 (and therefore exits the light delivery element 26). The semi-reflective material 52 permits a greater amount of incident light to pass through than does the fully reflective material 50. The specific degree to which the semi-reflective material 52 allows light to pass through can be varied to suit the application at hand. Typically, the degree to which the semi-reflective material 52 reflects light versus allowing light to pass through is chosen so that the light delivery element 26 produces the desired uniform distribution of light exiting the light delivery element 26.

The exemplary embodiment shown in FIGS. 10 and 10A includes protruding elements 40 extending outwardly from the scalp side surface 38 of the light delivery element 26. The semi-reflective material 52 may be disposed between the scalp side surface 38 and some or all of the protruding elements 40. In some embodiments (e.g., embodiments wherein the scalp side surface 38 is exposed between protruding elements 40—i.e., in the trenches 44), fully reflective material 50 may be disposed on the scalp side surface 38 between protruding elements 40; i.e., thereby restricting light exit only via the protruding elements 40. The inclusion of fully reflective material 50 on the side surfaces 46 and the back surface 48, and the semi-reflective material 52 on at least a portion of the scalp side surface 38 causes most light entering the light delivery element 26 to reflect multiple times within the light delivery element 26 before exiting the light delivery element 26. The multiple reflections produce a more uniform distribution of light exiting the light delivery element 26. The semi-reflective material 52 participates in these internal reflections, but also permits light to exit the light delivery element 26 via the protruding elements 40. As stated above, the degree to which the semi-reflective material 52 reflects light (or conversely allows light to pass through the material 52) can be altered to produce greater or lesser reflectivity internally within the light delivery element 26, which internal reflectivity is believed to influence the uniformity of light intensity exiting the light delivery element 26 via the protruding elements 40.

FIGS. 11 and 11A illustrate another exemplary light delivery element 26 embodiment. In this embodiment, the light delivery element 26 comprises a body of light conductive material that has a plurality of side surfaces 46 (e.g., three), a curvilinear back surface, and a scalp side surface 38. A light conduit (e.g., one or more optical fibers 34) operable to communicate light from the excitation light source 24 to the light delivery element 26 is shown connected to one of the side surfaces 46 of the light delivery element 26. A fully reflective material 50 may be disposed on the back surface 48 in the manner described above. The aforesaid fully reflective material 50 may also be disposed on the side surfaces 46. Excitation light directed into the light delivery element 26 from the side surface 46 (e.g., via the optical fibers 34) may be redirected from the curvilinear back surface 48 and toward the protruding elements 40 following a geometrical law based on the spatial light intensity distribution on the curved reflector and the desired spatial intensity distribution on the surface of the scalp.

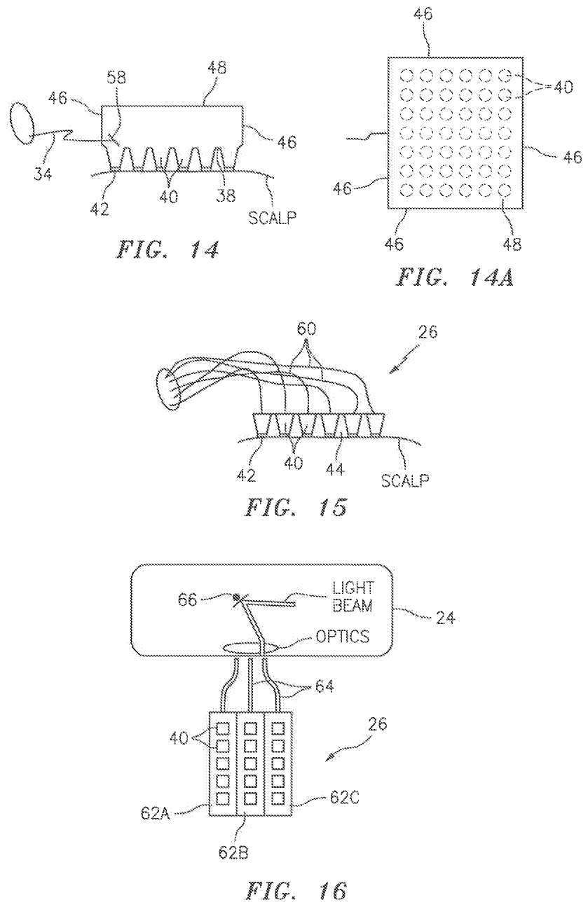

FIGS. 12 and 12A illustrate another exemplary light delivery element 26 embodiment. In this embodiment, the light delivery element 26 comprises a body of light conductive material that has a plurality of side surfaces 46 (e.g., three), a back surface 48, and a scalp side surface 38. The back surface 48 is disposed at an acute angle (“a”) relative to the scalp side surface 38. A light conduit (e.g., one or more optical fibers 34) operable to communicate light from the excitation light source 24 to the light delivery element 26 is shown connected to one of the side surfaces 46 of the light delivery element 26. A fully reflective material 50 may be disposed on each side surface 46 in the manner described above. A diffusive reflector 54 (e.g., a Fresnel reflector, or the like) that can be engineered to control the intensity distribution on the surface of the scalp is attached to the back surface 48; e.g., excitation light directed into the light delivery element 26 from the side surface 46 (e.g., via the optical fibers 34) may be incident to the diffusive reflector 54, which in turn controls the intensity distribution of light incident to the surface of the scalp via the protruding elements 40; e.g., thereby creating a uniform distribution of excitation light exiting the light delivery element 26 via the protruding elements 40.

FIGS. 13 and 13A illustrate another exemplary light delivery element 26 embodiment. In this embodiment, the light delivery element 26 comprises a body of light conductive material that has a plurality of side surfaces 46 (e.g., four), a back surface 48, and a scalp side surface 38. One or more light diffusive elements 56 are disposed within the body of the light delivery element 26. A fully reflective material 50 may be disposed on each side surface 46 and the back surface 48 in the manner described above. A light conduit (e.g., one or more optical fibers 34) operable to communicate light from the excitation light source 24 to the light delivery element 26 is shown connected to one of the side surfaces 46 of the light delivery element 26. Excitation light directed into the light delivery element 26 from the side surface 46 (e.g., via the optical fibers 34) is diffused by the one or more light diffusive elements 56. The one or more diffusive elements 56 create a uniform distribution of excitation light that exits the light delivery element 26 via the protruding elements 40.

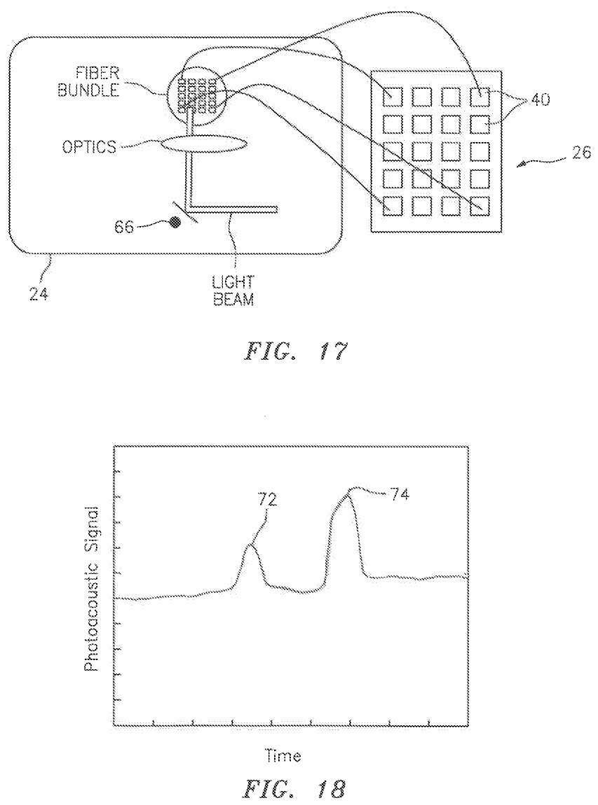

FIGS. 14 and 14A illustrate another exemplary light delivery element 26 embodiment. In this embodiment, the light delivery element 26 comprises a plurality of internal mirrors 58. A light conduit (e.g., one or more optical fibers 34) operable to communicate light from the excitation light source 24 to the light delivery element 26 is shown connected to one of the side surfaces 46 of the light delivery element 26. Excitation light directed into the light delivery element 26 from the side surface 46 (e.g., via the optical fibers 34) is reflected by the plurality of internal mirrors 58 to the respective protruding elements 40. The plurality of internal mirrors 58 help create a uniform distribution of excitation light that exits the light delivery element 26 via the protruding elements 40.

The light delivery elements 26 shown in FIGS. 10-14A are examples of light delivery elements 26 that are configured to provide a substantially uniform distribution of light intensity throughout the incidence region 36 of the light delivery element 26. These exemplary embodiments are provided to illustrate light delivery element 26 configurations. The present disclosure is not limited to these embodiments.

FIG. 15 illustrates another exemplary light delivery element 26 embodiment. In this embodiment, the light delivery element 26 includes a plurality of protruding elements 40 and optical fibers 60 providing a light conduit to each protruding element 40. The optical fibers may extend to the excitation light source 24, or may be connected to a manifold that connects to other light conduits (e.g., optical fibers 34).

In some embodiments of the present disclosure light delivery element 26, a light delivery element 26 may be configured to provide a non-uniform distribution of light intensity throughout the incidence region 36 of the light delivery element 26. In these embodiments, the incidence region 36 of the light delivery element 26 may be configured to have a plurality of incidence subregions; e.g., each incidence subregion having one or more protruding elements 40. At least one of these incidence subregions may be configured to provide a different light intensity distribution (e.g., higher) than other incidence subregions. For example, an incidence subregion that is positioned to cause the SSS, LTS, and/or the RTS (or portions thereof) to act as an acoustic aperture directed toward an ultrasonic receiver(s) 68 may be configured to provide excitation light at a greater light intensity (or a greater light intensity flux) than other subregions, and that greater light intensity (or flux) can improve the SNR.

In some embodiments of the present disclosure light delivery element 26, a light delivery element 26 may be configured to permit different incidence subregions of the light delivery to selectively receive light from the excitation light source 24; e.g., light from the excitation light source 24 may be delivered to some incidence subregions and not others, and the incidence subregions receiving light from the excitation light source 24 may be selectively switched to improve performance, etc. For example, not every subject will physically be the same (e.g., different head shapes, venous vessels located in different locations, etc.), or a light delivery element 26 may not always be properly positioned on a subject, or even if a light delivery element 26 was initially properly positioned on a subject, the light delivery element 26 may move (e.g., the subject moves the head apparatus 32) after the initial positioning. Embodiments of present disclosure light delivery elements 26 that enable an operator to selectively switch which incidence subregions are providing the excitation light (or greater or lesser amounts/intensities of excitation light) can permit the system operator to improve the performance of the system 22. In these embodiments, the controller 30 may be configured to execute instructions that cause the controller 30 to direct excitation light to the desired optical fibers and therefore the desired incidence subregions.

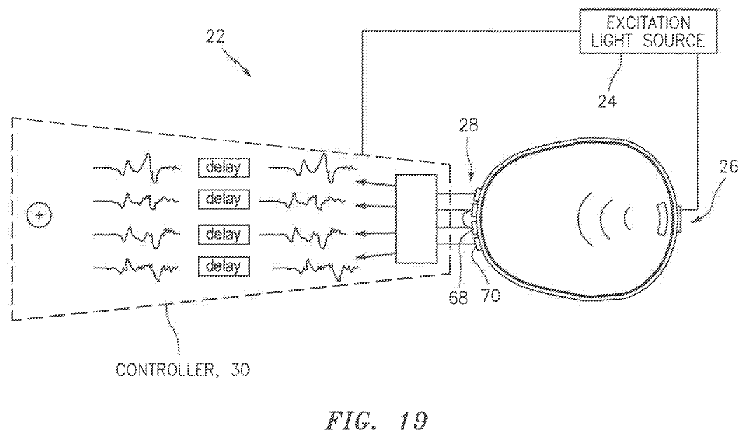

Referring to FIGS. 16 and 17, another example of a light delivery element 26 that is configured to permit different incidence subregions to selectively receive light from the excitation light source 24 is one having a controllable steerable mirror that is operable to direct the excitation light to selected incidence subregions. FIG. 16 diagrammatically illustrates an embodiment wherein a steerable mirror 66 is disposed with the excitation light source 24, and is controllable to steer the excitation light from the excitation light source 24 into incidence subregions 62A, 62B, 62C via optical fibers 64 (or other light conduits). Each incidence subregion 62A-62C may assume any of the light delivery element 26 configurations described above; e.g., one having fully reflective material affixed to side and back surfaces to produce a uniform light intensity distribution, or one having a curvilinear back surface or a diffusive surface proximate the back surface that redirects excitation light toward the scalp side surface 38 (and protruding elements 40 if included), or one having light diffusive elements disposed within the body of the light delivery element 26. FIG. 17 diagrammatically illustrates an embodiment wherein a steerable mirror 66 is disposed with the excitation light source 24, and is controllable to steer the excitation light from the excitation light source 24 into individual optical fibers (or other light conduits) within a fiber bundle to selectively communicate the excitation light to selectively chosen incidence subregions (e.g., individual protruding elements 40). Alternatively, a steerable mirror 66 (or other optics) may be disposed at the light delivery element 26, which mirror is operable to direct excitation light to a plurality of incidence subregions. In any of these embodiments that include a steerable mirror 66 (or the like), the steerable mirror 66 can be controlled (e.g., via instructions executed by the controller 30) to selectively direct excitation light to the desired incidence subregions.

As disclosed above, optical fibers (or other light conduits) may be used to communicate light from the excitation light source 24 to a side surface 46 of a light delivery element 26 embodiment. The side connection advantageously facilitates a low profile device for attachment to a subject. The present disclosure is not limited to light delivery elements 26 having a side connection to the excitation light source 24.

The ultrasound receiving mechanism 28 includes one or more ultrasonic receivers 68 (e.g., See FIGS. 1 and 2). As stated above, target analytes subjected to excitation light produce ultrasonic signals; e.g., typically in the wavelength range of 50 kHz to 3 MHz, but the present disclosure is not limited to this range. The ultrasonic receivers 68 are configured to sense the ultrasonic signals created by photoacoustic excitation of the target analyte(s) and to produce electronic signals representative thereof (referred to hereinafter as “sensor signals”). In preferred embodiments, the ultrasonic receivers 68 are configured to be placed in contact with the subject's forehead. The relative position of the subject's forehead and the posterior sections of the SSS, LTS and the RTS, make the forehead a preferred region for receiving the ultrasonic signals; i.e., the orientation of the ultrasonic receivers 68 at the subject's forehead is such that the sensed ultrasonic signals wavefronts are typically disposed substantially parallel to the ultrasonic receivers 68. The present disclosure is not, however, limited to ultrasonic receivers 68 configured to be placed in contact with the subject's forehead. Ultrasonic receivers 68 configured to be placed in contact with a subject's temple region, or near temple region can provide useful sensed information. In preferred embodiments, the ultrasonic receivers 68 configured to be disposed on the subject's forehead are large enough to receive a substantial portion of the ultrasonic signal wavefronts.

In some embodiments, the ultrasound receiving mechanism 28 includes one or more amplifiers 70 operable to amplify the electronic signals representative of the sensed ultrasonic signals; i.e., the sensor signals. An acceptable amplifier 70 would be a low noise amplifier configured for use with photoacoustic signals. The amplifiers 70 are preferably disposed in close proximity to the ultrasonic receivers 68 to minimize noise that may detrimentally affect the SNR of the ultrasonic receiving mechanism 28.

The instructions stored within a memory device in communication with the controller 30 (described below) are operable to distinguish detectable features from the sensed ultrasonic signals produced by the photoacoustically excited target analytes. FIG. 18 is a diagrammatic graph of photoacoustic signal (Y-axis) versus time (X-axis) at a single excitation wavelength to illustrate an initial ultrasonic signal peak 72 attributable to the ultrasonic signals produced by excitation light interrogation of the SSS, and a subsequent peak 74 attributable to the ultrasonic signals produced by excitation light interrogation of the subject's skull, scalp, and or skin. The graph is provided to illustrate the relative positions of the SSS peak 72 and the skin peak 74 (i.e., SSS peak 72 precedes skin peak 74), and to illustrate that the SSS peak 72 is distinguishable and readily trackable.

Referring to FIG. 19, in those system 22 embodiments that include a plurality of ultrasonic receivers 68, the output of each ultrasonic receiver 68 may be configured as a channel for processing purposes within the controller 30. The photoacoustically produced ultrasonic signals emanating from the light interrogation of target analytes disposed within the SSS, LTS, and RTS can arrive at different times at each of the ultrasonic receivers 68. Since these ultrasonic signals are identifiable (e.g., as described above with respect to FIG. 18), the time differences between respective ultrasonic signals can be accounted for within the stored instructions executed by the controller 30; e.g., FIG. 19 diagrammatically illustrates a “delay” function that operates to account for the signal time differences. For example, the ultrasonic signals (or more accurately, the signals produced by the ultrasonic receivers 68 sensing the ultrasonic signals) can be sensed and identified, the signal arrival time differences at the respective ultrasonic receivers 68 accounted for, and the aforesaid signals summed to determine the desired analyte parameter (e.g., concentration). The aforesaid steps are a non-limiting example of how the ultrasonic signals may be processed to provide a strong SNR.

The controller 30 is in communication with the excitation light source 24 and the ultrasound receiving mechanism 28 and other components (e.g., the light delivery element 26 in some embodiments) to perform the functions described herein. The controller 30 may include any type of computing device, computational circuit, processor(s), CPU, computer, or the like capable of executing a series of instructions that are stored in memory. The instructions may include an operating system, and/or executable software modules such as program files, system data, buffers, drivers, utilities, and the like. The executable instructions may apply to any functionality described herein to enable the system 22 to accomplish the same algorithmically and/or coordination of system 22 components. The controller 30 may include a single memory device or a plurality of memory devices. The present disclosure is not limited to any particular type of non-transitory memory device, and may include read-only memory, random access memory, volatile memory, non-volatile memory, static memory, dynamic memory, flash memory, cache memory, and/or any device that stores digital information. The controller 30 may include, or may be in communication with, an input device that enables a user to enter data and/or instructions, and may include, or be in communication with, an output device configured, for example to display information (e.g., a visual display or a printer), or to transfer data, etc. Communications between the controller 30 and other system 22 components may be via a hardwire connection or via a wireless connection.

Both the light delivery element 26 and the ultrasound receiving mechanism 28 (or at least portions thereof) may be attached to the head apparatus 32. The head apparatus 32 may be configured to locate the light delivery element 26 and the ultrasound receiving mechanism 28 relative the subject's head and maintain them in communication with the subject's head during sensing. The head apparatus 32 may assume a variety of different forms (e.g., a headband, hat, cap, harness, appliance, or the like). The present disclosure is not limited to any particular head apparatus 32 configuration. The head apparatus 32 may be adjustable to fit the subject's head. Embodiments of the head apparatus 32 may be configured to have a relatively short “height” (i.e., the maximum distance that the apparatus extends out from the subject's scalp along a line perpendicular to the scalp) which may also be described as having a “low profile”. The low profile is believed to be more comfortable for the subject to wear, less likely to be interfered with (e.g., contact external surfaces), and more likely to maintain its initial position after attachment to the subject's head. As described above, in some embodiments the light delivery elements 26 are configured to receive excitation light from optical fibers (or other light conduits) extending out from a side surface of the light delivery element 26 body. Placing the optical fibers (and electrical leads as may be included) on a side surface 46 is helpful in producing a low profile head apparatus 32. In some embodiments, the head apparatus 32 may be configured so that the relative positions of the light delivery element 26 and the ultrasound receiving mechanism 28 (more specifically the positions of the light delivery element 26 and each of the ultrasound receivers, and the relative positions of the ultrasound receivers) are fixed or adjustable.

Embodiments of the head apparatus 32 are configured to facilitate alignment of the light delivery element(s) 26 with the SSS, LTS, or RTS, and alignment with the ultrasonic receivers 68. Useful signal information can be acquired even if there is some degree of misalignment between the light delivery element(s) 26 and the subject's SSS, LTS, or RTS, and/or some degree of misalignment between the light delivery element(s) 26 and the ultrasonic receivers 68. However, alignment (as defined herein) between the light delivery element(s) 26, the subject's SSS, LTS, or RTS, and the ultrasonic receivers is understood to improve the SNR. The various head apparatus 32 configurations promote more accurate alignment than, for example, systems that require the user to subjectively apply a light source and ultrasonic receivers to a subject's head independent of one another.

In some embodiments, the positive or negative contribution to the SNR of the sensed signals from each channel may be evaluated. One or more channels receiving weak ultrasound signals may have a low SNR relative to other channels, and conversely one or more channels receiving strong ultrasound signals may have a high SNR relative to other channels. Hence, including signals from a “weak channel” may adversely affect the overall SNR of the sensed signals, whereas including signals from a “strong channel” may positively affect the overall SNR of the sensed signals. Signals from a weak channel may be a result of the placement of the ultrasonic sensor associated with the channel, or because the ultrasonic sensor associated with the channel has dislodged or is not properly attached, or the like.

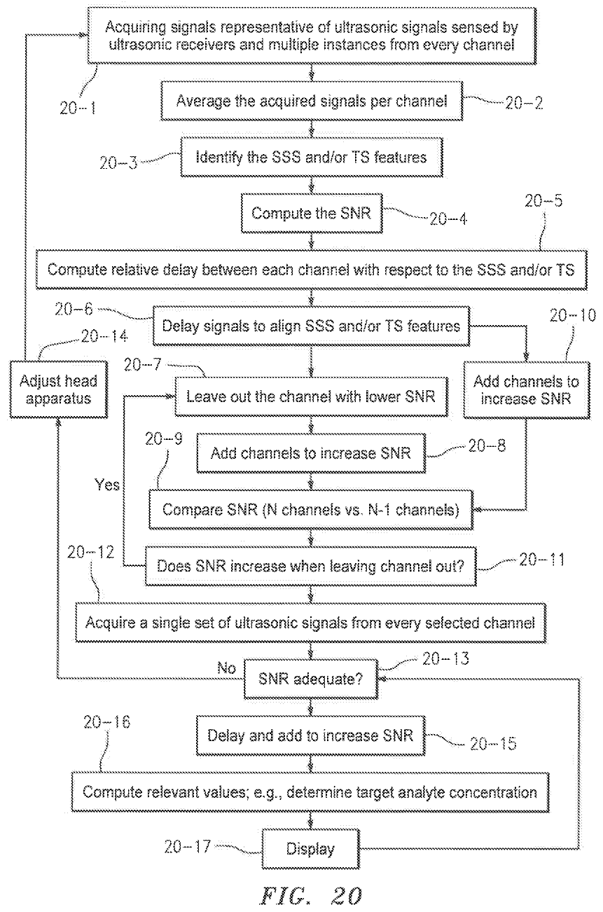

FIG. 20 illustrates a flow chart of an example algorithm that may be used according to the present disclosure. This exemplary algorithm includes steps that address the relative signal and SNR contributions from “weak” and “strong” channels, ultimately providing the system 22 with an improved SNR. Stored instructions when executed cause the controller 30 to perform most of the described steps, although some of the described steps may be optional and therefore not required. The order of the steps within the flow chart represent one embodiment of how the steps may be performed. The present disclosure is not limited to the specific order shown.

In Step 20-1, the excitation light source 24 is operated to provide excitation light multiple times. The excitation light causes photoacoustic responses/ultrasonic signals to be emitted from those target analytes present within the venous blood source targeted (e.g., SSS, TS). The ultrasonic signals are sensed and the sensor signals produced multiple times for each channel.

In Step 20-2, the sensor signals may be averaged. The term “average” as used here contemplates that a number is produced that is representative of the sensed signals produced each of the multiple times for a given channel. The term “average” is not limited to an arithmetic mean (i.e., the sum of the numbers divided by how many numbers are being averaged), and alternatively may be another measure of central tendency, such as a mean, median, or mode value, or the like.

In Step 20-3, the multiple instance sensor signals within a channel (e.g., the averaged signals) are analyzed to determine relevant features (e.g., features/peaks associated with the SSS and/or TR).

In Step 20-4, the SNR for each channel is determined based on the multiple instance sensor signals within that channel (e.g., the averaged signals).

In Step 20-5, values are determined representative of the relative sensor signal arrival time delay between channels.

In Step 20-6, values are determined representative of the determined relative delays to align the SSS and/or TS features for each sensed signal channel.

In Step 20-7, the SNR values for the channels are analyzed and one or more channels with a low SNR (relative to the other channels) may be removed from the algorithmic analysis.

In Steps 20-8 and 20-10, sensed signals from additional channels may be added to potentially increase the SNR.

In Step 20-9, the collective SNR from a first grouping of channels (e.g., “N” number of channels, where “N” is an integer) may be determined, and the collective SNR from at least one additional grouping of channels (e.g., “N-1” number of channels) may be determined and the determined SNR values compared. If the collective SNR value increases when a low SNR channel is removed, then pursuant to Step 20-11 the process may return back to Step 20-7, and the low SNR value channel removed. A converse process may be used to evaluate whether adding one or more channels would improve the collective SNR; e.g., see Steps 20-8 and 20-10.

Once a grouping of channels is determined believed to have an acceptable SNR, the system 22 may then acquire sensor signals from each channel in the grouping based on ultrasonic signals collected at, or nearly, the same point in time (albeit subject to the delays; e.g., see Step 20-12). The SNR of that grouping of channels may then be determined to verify the collective SNR is acceptable; e.g., see Step 20-13. If the collective SNR from the grouping is not acceptable, then the process may begin again at Step 20-1. Also, if the collective SNR from the grouping is not acceptable, then the system 22 may be configured to produce a message to the operator that the head apparatus 32 on the subject may need adjustment; e.g. see Step 20-14.

If the collective SNR from the grouping is determined to be acceptable (e.g., within an acceptable range), then the sensor signals acquired from each channel in Step 20-12 may be evaluated for relative time delays between channels, and sensor signals from additional channels added as appropriate; e.g., see Step 20-15. The sensor signals may then be used to determine relevant values relating to the target analytes (e.g., HbO2 concentration, Hb concentration, etc.) and/or venous blood parameters (e.g., oxygen saturation) utilizing the collected target analyte information.

To be clear, the present disclosure is not limited to the methodology described above and shown in the flow chart of FIG. 20. The aforesaid example is provided to illustrate the utility of the present disclosure and not to limit the present disclosure.

While various inventive aspects, concepts and features of the disclosures may be described and illustrated herein as embodied in combination in the exemplary embodiments, these various aspects, concepts, and features may be used in many alternative embodiments, either individually or in various combinations and sub-combinations thereof. Unless expressly excluded herein all such combinations and sub-combinations are intended to be within the scope of the present application. Still further, while various alternative embodiments as to the various aspects, concepts, and features of the disclosures—such as alternative materials, structures, configurations, methods, devices, and components, alternatives as to form, fit, and function, and so on—may be described herein, such descriptions are not intended to be a complete or exhaustive list of available alternative embodiments, whether presently known or later developed. Those skilled in the art may readily adopt one or more of the inventive aspects, concepts, or features into additional embodiments and uses within the scope of the present application even if such embodiments are not expressly disclosed herein.

Additionally, even though some features, concepts, or aspects of the disclosures may be described herein as being a preferred arrangement or method, such description is not intended to suggest that such feature is required or necessary unless expressly so stated. Still further, exemplary or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of exemplary methods or processes are not limited to inclusion of all steps as being required in all cases, nor is the order that the steps are presented to be construed as required or necessary unless expressly so stated. The words used in the claims have their full ordinary meanings and are not limited in any way by the description of the embodiments in the specification.

What is claimed is:

1. A system for measuring one or more analytes within cerebral venous blood flow, comprising:
an excitation light source configured to selectively produce one or more wavelengths of excitation light, the one or more wavelengths of excitation light operable to cause an analyte present within venous blood flow to photoacoustically produce at least one ultrasonic signal;
a light delivery element configured to be disposed on a posterior region of a human head, the light delivery element configured to receive the excitation light from the excitation source, the light delivery element having a scalp side surface and a plurality of protruding elements extending out from the scalp side surface, the protruding elements configured as light conduits, and are spaced apart from one another;
an ultrasound receiving mechanism configured to be disposed on an anterior region of the human head, the mechanism including at least one ultrasonic receiver disposed to sense the at least one photoacoustically produced ultrasonic signal and produce a sensed signal representative of the photoacoustically produced ultrasonic signal; and
a controller in communication with the excitation light source and the ultrasound receiving mechanism, and a memory storing instructions, the instructions when executed cause the controller to:
control the excitation light source to selectively produce the one or more wavelengths of excitation light; and
measure an amount of the analyte present within the cerebral venous blood flow using the sensed signals produced by the at least one ultrasonic receiver, the sensed signals representative of the photoacoustically produced ultrasonic signals.

2. The system of claim 1, wherein the light delivery element is configured to distribute the excitation light in a substantially uniform manner through the protruding elements.

3. The system of claim 2, wherein the light delivery element includes a plurality of side surfaces, and a back surface opposite the scalp side surface, and a fully reflective material attached to the plurality of side surfaces and to the back surface, the fully reflective material disposed to reflect the excitation light internally within the light delivery element.

4. The system of claim 3, wherein the light delivery element further includes a semi-reflective material attached to the scalp side surface, the semi-reflective material disposed to reflect less than all of the excitation light incident to the semi-reflective material internally within the light delivery element.

5. The system of claim 2, wherein the light delivery element includes a plurality of side surfaces, and a back surface opposite the scalp side surface, the back surface have a curvilinear shape configured to direct excitation light incident to the back surface internally within the light delivery element towards the protruding elements.

6. The system of claim 2, wherein the light delivery element includes a plurality of side surfaces, and a back surface disposed at an acute angle relative to the scalp side surface, and a diffusive reflector disposed proximate the back surface, the diffusive reflector configured to reflect excitation incident to the diffusive reflector internally within the light delivery element towards the protruding elements in a uniform distribution.

7. The system of claim 1, wherein the light delivery element has an incidence region that includes the plurality of protruding elements, wherein the incidence region is configured to permit alignment with at least a portion of a superior sagittal sinus, and at least a portion of a transverse sinus.

8. The system of claim 7, wherein the incidence region is configured to permit alignment with at least a portion of the superior sagittal sinus, and at least a portion of a left transverse sinus or at least a portion of a right transverse sinus.

9. The system of claim 8, wherein the incidence region is configured to permit alignment with at least a portion of the superior sagittal sinus, and at least a portion of both the left transverse sinus and the right transverse sinus.

10. The system of claim 1, further comprising a head apparatus configured to support the light delivery element and ultrasound receiving mechanism, the head apparatus configured to position the light delivery element on a posterior region of a human head, and configured to position at least a part of the ultrasound receiving mechanism on an anterior region of the human head, the light delivery element having an incidence region configured to permit alignment with at least a portion of a superior sagittal sinus, and at least a portion of a left transverse sinus or at least a portion of a right transverse sinus.

11. The system of claim 1, wherein the light delivery element has a plurality of incidence subregions, the incidence subregions disposed to permit alignment with at least a portion of a superior sagittal sinus, and at least a portion of a transverse sinus, and wherein the instructions when executed cause the controller to direct the excitation light to select ones of the plurality of incidence subregions.

12. A method for measuring one or more analytes within cerebral venous blood flow of a subject, comprising:
using an excitation light source to produce one or more wavelengths of excitation light, the one or more wavelengths of excitation light operable to cause an analyte present within venous blood flow to photoacoustically produce at least one ultrasonic signal;
directing the excitation light to a posterior region of a human head using a light delivery element, the excitation light oriented to be incident to at least a portion of a superior sagittal sinus of the subject, at least a portion of a left transverse sinus, or at least a portion of a right transverse sinus of the subject;
using an ultrasound receiving mechanism to sense ultrasonic signals from an anterior tissue region of the subject, and to produce sensed signals representative of the sensed ultrasonic signals; and
using a controller in communication with the excitation light source and the ultrasound receiving mechanism to measure an amount of the analyte present within the at least a portion of the superior sagittal sinus of the subject, the at least said portion of said left transverse sinus, or the at least said portion of said right transverse sinus of the subject using the sensed signals produced by the ultrasound receiving mechanism.

13. The method of claim 12, wherein the step of directing the excitation light to the posterior region of the human head, includes directing at least a portion of the excitation light to be incident to the at least said portion of the superior sagittal sinus of the subject, the at least said portion of said left transverse sinus, or at least said portion of said right transverse sinus in a manner that creates an acoustic aperture directed substantially toward the ultrasonic receiving mechanism.

14. The method of claim 12, wherein the step of directing the excitation light to said posterior region of said human head using said light delivery element, includes directing the excitation light to said posterior region in a substantially uniform distribution of light intensity.

15. The method of claim 12, wherein the light delivery element has a plurality of incidence subregions, and the step of directing the excitation light to said posterior region of said human head using said light delivery element, includes directing the excitation light to at least a first one of the plurality of incidence subregions, and not directing the excitation light to at least a second one of the plurality of incidence subregions.

16. The method of claim 15, directing at least a portion of the excitation light to be incident to the at least said portion of the superior sagittal sinus of the subject, the at least said portion of said left transverse sinus, or at least said portion of said right transverse sinus in a manner that creates an acoustic aperture directed substantially toward the ultrasonic receiving mechanism.

17. The method of claim 12, wherein the step of using said controller to measure said amount of said analyte includes determining a signal to noise ratio (SNR) for each of a plurality of channels.

18. The method of claim 17, wherein the step of using said controller to measure said amount of said analyte includes measuring said amount of said analyte with said sensed signals from less than all of said channels.

19. The method of claim 17, wherein the step of using said controller to measure said amount of said analyte includes determining a time delay of the sensed signals between the respective channels.

20. The method of claim 12, wherein the step of using said controller to measure said amount of said analyte includes using the sensed signals to identify at least one of the superior sagittal sinus, the left transverse sinus, or the right transverse sinus.
